(19) United States
(12) Patent Application Publication
(10) Pub. No.: US 2020/0121904 A1
(43) Pub. Date: Apr. 23, 2020

(54) REVERSIBLE ROTARY TATTOO MACHINE WITH CENTRIC CAM

(71) Applicant: Daniel Kubin, Independence, MO (US)

(72) Inventor: Daniel Kubin, Independence, MO (US)

(21) Appl. No.: 16/666,009

(22) Filed: Oct. 28, 2019

Related U.S. Application Data
(63) Continuation-in-part of application No. 16/164,735, filed on Oct. 18, 2018.

(51) Int. Cl. A61M 37/00

(52) U.S. Cl. CPC A61M 37/0076

(57) ABSTRACT

The present invention provides an improved reversible rotary tattoo machine, wherein in one aspect a machine is provided that in some embodiments will present a reversible motor coupled to a housing and a linking member secured to the housing along with a drive element received by the housing which is in pivoted communication with the linking member where the drive element includes at least a first rotating member having an asymmetrical outer surface which oscillates the drive element during operation of the reversible motor.

### CROSS-REFERENCE TO RELATED APPLICATION

This application is a non-provisional application claiming the benefit of the prior filed U.S. provisional application No. 62/574,472, filed Oct. 19, 2017 the contents of which are incorporated herein as part of the file wrapper and the electronic records at the United States Patent Office.

### FIELD OF THE INVENTION

The present invention is broadly directed to tattoo machines and more specifically to a reversible rotary tattoo machine with an improved housing and cam which provides an improved stroke cycle.

### BACKGROUND OF THE INVENTION

Tattoos are typically applied by a tattoo artist using a tattoo machine to reciprocally drive a needle assembly into the skin and deliver pigment under the skin. The reciprocating movement of the needle assembly may be achieved using a pneumatic, hand-held coil or rotary tattoo machine. The pneumatic machine is air powered while the other machines are electrically powered with an electrical power supply and typically operated by the artist through the use of a foot switch. Rotary tattoo machines convert the rotary movement of an electrical motor into linear cycle where the need oscillates in an up and down pattern.

Tattoo artists create a tattoo by implanting the desired amount of ink during the needle stroke to produce the desired effect. The typical needle stroke involves By way of example, and not as a limitation, a conventional tattoo machine may operate, for example, between 60 to 120 cycles per send, a stroke length between 2 mm and 5 mm with an average length of about 3.5 mm millimeters and a force of approximately half a pound which is tested by feeling the resistance of an armature while running by placing a thumb against the armature. A typical setting is approximately 100 cycles, 2.5 millimeters and half a pound of pressure.

Conventional tattoo machine are not easily tunable. Tattoo machine operating parameters include frequency, i.e., number of needle strokes per unit of time, speed or velocity of the needle and amplitude or depth which a needle penetrates. Adjusting a machine often requires adjusting one mechanical element one at a time because each impacts the others so it can be a difficult and arduous task, if not nearly impossible. In order for the conventional tattoo machine to work smoothly, it may require careful tuning by an experience tattoo operator prior to use and must be maintained to provide the desired operating characteristics.

A conventional tattoo machine operates a needle in a linear manner through a series of push and pull motion in which the armature and needle are reciprocally driven up and down. The cycle the needle goes through is typically referred to as the needle stroke. In some tattoo machines, the reciprocal movement is symmetrical in that the upward movement is identical to the downward movement and the linear movement is consistent throughout the stroke. In some rotary machines, the linear cycle slows down dramatically at the bottom of the needle stroke. However, it is more difficult to pierce the skin precisely and efficiently when the needle speed has slowed. Being able to puncture the skin precisely and efficiently is crucial for tattoo tasks such as lining or detailed shading associated with tattoo work. There is a need for a tattoo machine which provides an improved needle stroke which pierces the skin precisely and efficiently.

The needle assembly typically includes multiple needles and the shape, size, and arrangement of needles are selected depending on the effect or design desired for the tattoo. For a rotary tattoo machine, the needle configuration, as well as the configuration of the rotary tattoo machine's motor, stroke wheel, and slide, all may be varied either within a single machine assembly or system, or between different machines. Generally, the “hit” of the tattoo machine is based upon the stroke length with some rotary tattoo machines having a fixed stroke, others an adjustable stroke. In some cases, a rotary machine may provide for a “give” adjustment that softens the hit, however, these machines still have the disadvantage of not allowing for ease of pigment delivery. Typically, the cam cycle of these machines slows the needle down at the bottom of the stroke, increasing the time the needle is inserted into the skin which causes an increased discomfort during the tattoo process. Therefore, there is a need to provide an improved rotary tattoo machine with a softer hit which provides for easier pigment delivery where the “hit” is variable and is not based simply upon the stroke length and allows for manipulation of the needle puncture time without tuning or changing our various components.

In addition, as previously indicated, often rotary tattoo machine components must be manually varied to provide the desired operational characteristics which different artists require to use the machine for different tattooing processes like, lining, coloring, or shading steps in the tattooing process. Often this requires adjusting the stroke or changing out the cam to obtain the desired stroke length, which some machines do not allow. Therefore, there is a need for an improved tattoo machine which allows for easily modifying and varying the machines' operational characteristics such as the needle stroke without changing out various components.

Some rotary tattoo machines include a return spring which helps return the drive element of the machine, and the attached needle, to its resting or up position while the machine is in use, and particularly after use is stopped. Some machines use a cartridge needle which includes a disposable tip with a self-contained needle. In some cases, these cartridge needles have a built-in return bias which causes spring tensioned tuned machines to malfunction. Therefore, there is a need to provide a tattoo machine with adjustable bias which allows for easily manipulation during operation in a forward or reversible operation and allows for varying stroke characteristics while maintaining the desired speed and efficiency, without removing or changing lots of components.

Accordingly, there is a need for a more efficient tattoo machine which allows for faster and more precise work which allows for quicker recovery periods and for more tattooing, either in terms of area covered or number of tattoos, which can be accomplished by the artist within in a given time period which addresses at least a portion of the aforementioned shortcomings.

### SUMMARY OF THE INVENTION

According to one embodiment of the present invention there is provided an improved rotary tattoo machine which includes An improved rotary tattoo machine with a reversible motor, said rotary tattoo machine comprising a housing, a linking member secured to said housing, a drive element received by said housing and in pivoted communication with said linking member; and said drive element further comprising at least a first rotating member having an asymmetrical outer surface which oscillates said drive element during operation of said reversible motor.

In accordance with another embodiment of the present disclosure there is provided a rotary tattoo machine including a linking member configured to engage a tattoo needle assembly; a body, the body defining a first cavity for pivotal engagement of the linking member, and the lower portion of the body presenting a channel from an exterior surface of the lower portion of the body to the first cavity, the channel being configured for selective adjustment of a drive element, said drive element including at least a first rotating member having an asymmetrical outer surface; a motor in mechanical communication with the drive element, the motor causing the drive element to rotate and oscillate which causes the linking member to pivot upward and downward in a non-undulating manner.

Another embodiment of the present invention is a rotary tattoo machine with a rotating cam assembly engaged by a follower arm that provides stability for the rotating cam assembly as it rotates the swing arm between an upward and a downward position, the rotating cam assembly having an asymmetrical cam lobe which can alternate the upward and downward thrust characteristics of the “hit” based upon the polarity of the motor which is changed by varying the electrical connectivity at a pair of terminals.

Another embodiment of the invention is a rotary tattoo machine with an alternative rotating cam assembly which is stabilized as it rotates in an offset manner between an upward and downward position, the rotating cam assembly having an actuator which can alternate the upward and downward thrust characteristic of the “hit.” The rotating cam assembly includes a spring embedded cam with a retractable spring which provides a dampener for shifting the cam bearing during rotation.

In one feature of the invention, the return spring tension or bias may be adjusted (for softer or stiffer operation) without removal to provide the desired bias that the artist prefers (which may be softer or stiffer), and that provides greater precision over the return spring tension all while the motor remains coupled to the slide member. Alternatively, for use of the machine with a tube and needle assembly or cartridge having an integrated biasing means to move a needle of the needle assembly to a return position within the tip or cartridge, the tension on the return spring may be removed entirely through the use of an adjustment screw accessible through the channel while the motor remains coupled to the slide member.

In some embodiments, there is provided a system including the tattoo machine described above, the return spring, a retaining means, a needle assembly and a tube having a stem, a grip and a tip. The system may include a set of return springs, each return spring in the set having a different compression rating. In some embodiments, there is provided a system including the tattoo machine described above, and a stem, a grip, a needle drive bar and a needle cartridge which has a tip and one or more tattoo needles. The cartridge may include a bias means to move a needle of the needle assembly to a return position within the cartridge. Either system may be provided with a set of return springs having a range of compression ratings, or other characteristics, in order to allow a tattoo artist to control and configure the feel and performance of the system.

Certain embodiments of the invention are outlined above in order that the detailed description thereof may be better understood, and in order that the present contributes to the art may be better appreciated. There are, of course, additional embodiments of the invention that will be described below and which will form the subject matter of any claims appended hereto.

In this respect, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein as well as the abstract are for the purposes of description and should not be regarded as limiting.

As such, those skilled in the relevant art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention. Though some features of the invention may be claimed in dependency, each feature has merit when used independently.

Various objects and advantages of the present invention will become apparent from the following description taken in conjunction with the accompanying drawings wherein are set forth, by way of illustration and example, certain embodiments of this invention. The drawings submitted herewith constitute a part of this specification, include exemplary embodiments of the present invention, and illustrate various objects and features thereof.

### BRIEF DESCRIPTION OF THE DRAWINGS

Further features of the present invention will become apparent to those skilled in the art to which the present invention relates from reading the following description with reference to the accompanying drawings, in which a better understanding of the present invention is depicted, in which:

FIG. 1A is a depiction of a prior art bearing arrangement and resultant wave form representing operation of a prior art rotary tattoo machine.

FIG. 1 is a front perspective of an exemplary embodiment of the present improved tattoo machine with optional accessories.

FIG. 2 is a front elevation of the exemplary embodiment of an improved tattoo machine illustrated in of FIG. 1.

FIG. 3 is a partial exploded view of an exemplary motor, cam and swing arm in accordance with one embodiment of the invention illustrated in FIG. 1.

FIG. 4 is a cross section of the exemplary swing arm in accordance with the invention illustrated in FIG. 1.

FIG. 5 is a back elevation of an exemplary swing arm in accordance with an embodiment of the invention illustrated in FIG. 1 with the cam in a neutral orientation.

FIG. 6 is a back elevation of the exemplary swing arm in accordance with the swing arm embodiment of the invention illustrated in FIG. 4 with the cam in an upward orientation.

FIGS. 7-8 are top plan views of an exemplary embodiment of the cam.

FIG. 9 is a front perspective of a second embodiment of an improved tattoo machine.

FIG. 10 is a rear perspective of the second embodiment of the improved tattoo machine according to FIG. 9.

FIG. 11 is a rear exploded view of the second embodiment of the improved tattoo machine according to FIG. 9.

FIG. 12 is a cross-sectional view of an alternative swing arm in accordance with the improved tattoo machine embodiment of FIG. 9 in the lower position.

FIG. 13 is the cross-sectional view of the alternative swing arm of FIG. 12 in the upper position.

FIG. 14 is a side cross-sectional view of the alternative embodiment of FIG. 9 with the alternative embodiment of the swing arm of FIG. 12.

FIG. 15 is a partially exploded view of one aspect of the reversible rotary prior art tattoo machine in accordance with FIG. 9.

FIG. 16 is an graphical illustration of the resultant wave form generated during operation of the improved tattoo machine according to FIG. 9.

FIG. 17 is a partially exploded view of an alternative cam assembly.

FIG. 18 is a side cross-sectional view of the alternative cam assembly of FIG. 17.

FIG. 19 is a front cross-sectional view of the alternative cam assembly of FIG. 17.

FIG. 20 is a graphical illustration of the resultant wave form generated during operation of the improved tattoo machine with alternative cam assembly of FIG. 17.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The problems identified above and difficulties associated therewith are obviated, at least in part, by the present invention which relates generally to an improved rotary tattoo machine design. More specifically, the present invention is a reversible rotary-type tattoo machine which utilizes a non-symmetrical cam to increase or decrease the acceleration associated with the tattoo needle to provide a softer or harder hit of the needle impacting/penetrating the skin. Reversing the polarity of the motor modifies the operation of the needle to reduce or increase the needle travel speed which effects the impact of the needle upon the surface of the skin and in this way assists the operator in modifying the tattooing characteristics. Reference numeral 20 generally refers to an embodiment of the present invention, a reversible rotary tattoo machine. Reference numeral 120 generally refers to a second embodiment of the present invention.

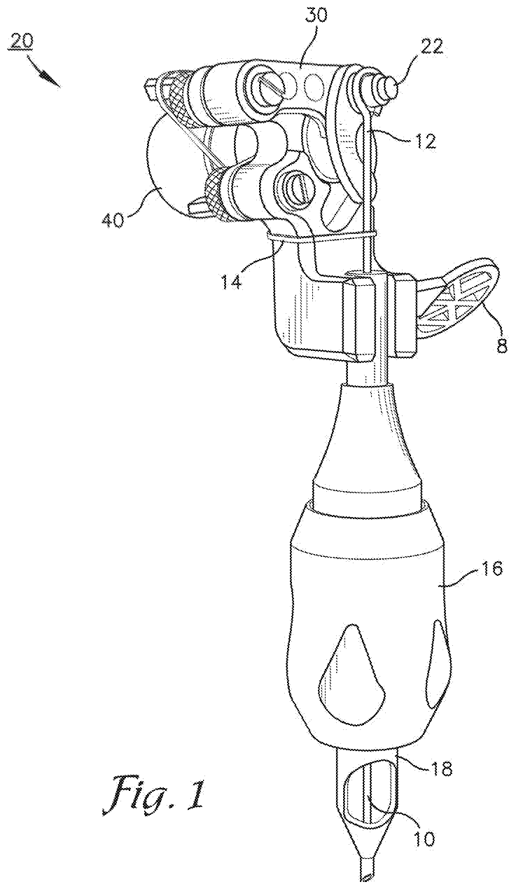

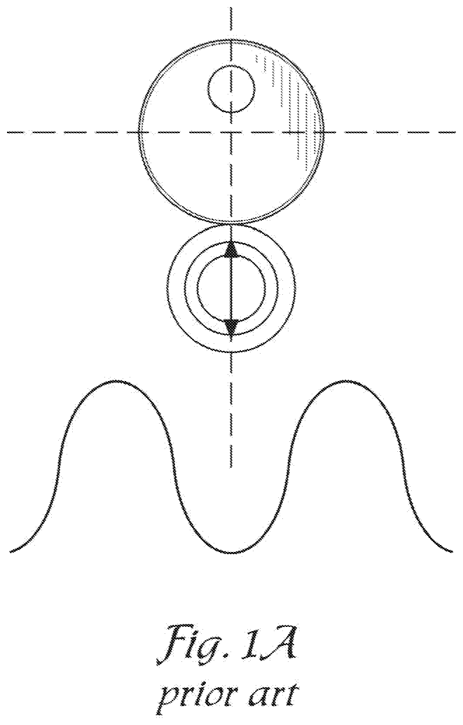

Prior art rotary tattoo machines typically move a needle up and down in a uniform manner with the penetration of the needle into the skin occurring typically as the needle moves down and it leaves the skin when the needle moves up. The oscillation is uniform and undulating. The time at the top and bottom of the up and down cycle can seem elongated because the needle is moving the slowest at the top and bottom of the cycle. As the slow moving needle penetrates and remains under the skin pain and discomfort may be felt by the person receiving a tattoo with the prior art machine. In addition, reversing the direction of the rotation, clock wise or counter clock wise will result in the same movement. FIG. 1A depicts an arrangement and resulting motion which may be generated by a representative prior art rotary tattoo machine in which the rotation of a bearing in an offset manner about another bearing creates the symmetrical, undulating up and down motion generated by some prior art rotary tattoo machines.

FIG. 1 depicts an embodiment of the reversible rotary tattoo machine 20 in operation with a tattoo needle 10 connected to the machine 20 through an armature 12 and further including a grip 16 connectably secured to a cartridge 18 during operation of the tattoo needle 10 by the tattoo machine 20. The armature 12 may include, but is not limited to, a needle bar or other known mechanical linking members which connect between the drive element 22 and the needle 10, allowing for communication between the motor 40 and the needle 10. The tattoo machine 20 includes a pair of terminals 42 spaced along housing 24 and in electrical communication with the rotary motor 40. The tattoo machine 20 further includes a drive element 22 in communication with the tattoo needle 10 through the armature 12 which is received by an adjustable armature receiver 26 which is selectively adjustable by, for example, the depicted thumbscrew 28 or with other generally known mechanical adjustment mechanisms. In addition, a resilient band 14 as depicted in FIG. 1 may be utilized to maintain, retain and/or stabilize the armature 12 as desired.

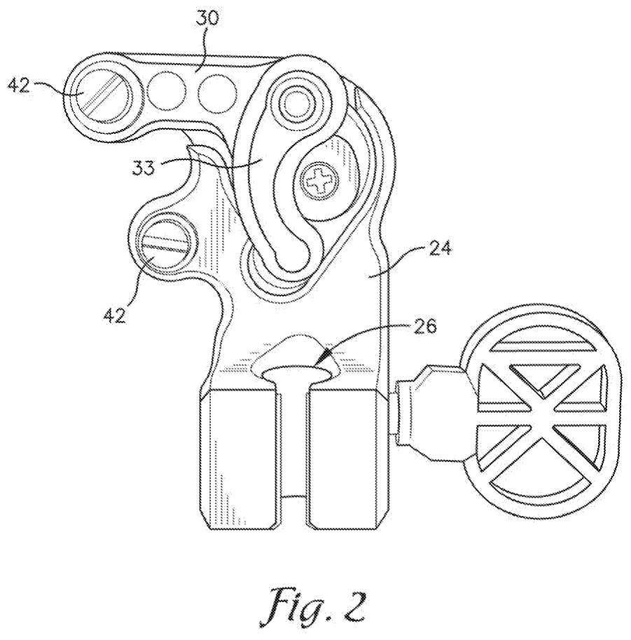

FIG. 2 illustrates the reversible rotatory tattoo machine 20 with the swing arm 30 connectably secured to the motor 40 through the housing 24. In operation, the swing arm 30 angularly pivots up and down, as the motor 40 rotates. The angular motion of the swing arm 30 varies, in part, based on the polarity of motor 40 which is modified for example by changing the polarity of the energized terminals 42 from, by example, alternating the power source leads.

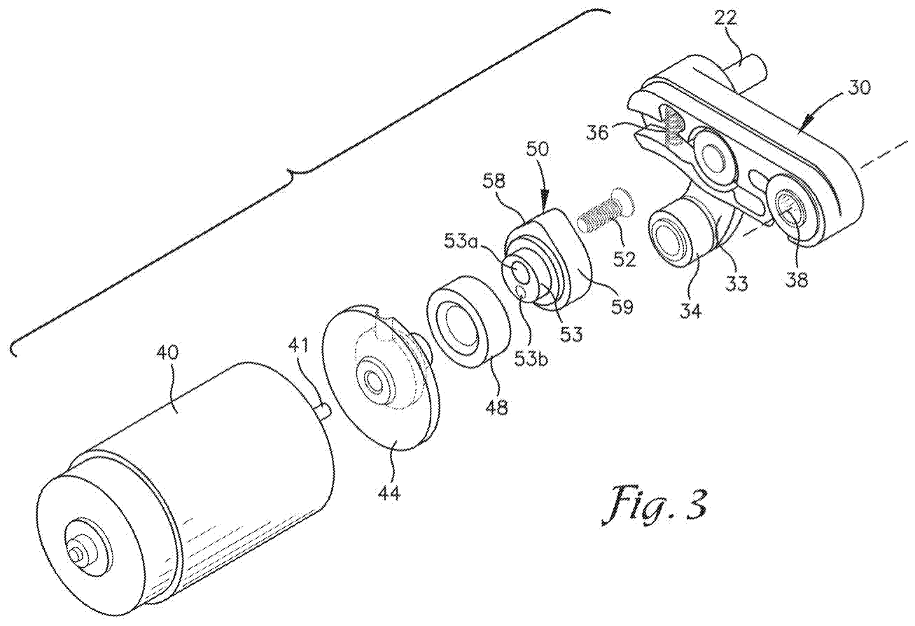

As further illustrated in FIG. 3, the depicted embodiment of the swing arm 30 has a horizontally extending arcuate body with a downward depending pivot arm 33, the swing arm 30 being generally offset in relation to the motor 40 and adapted for upward and downward pivoted rotation about a pivot point 38 during operation. The swing arm 30 is depicted with a generally arcuate horizontal structure with an outwardly biased following arm 32 and a downwardly depending convex pivot arm 33. The following arm 32 is spaced from the swing arm 30 with biasing member 36 which allows for inward compression and outward decompression of the following arm 32 during operation. In addition, the depicted embodiment of the swing arm 30 includes a plurality of compressible structures which allow for additional compression of the swing arm 30. The pivot arm 33 includes a swing arm bearing 34.

The swing arm 30 in conjunction with the reversible motor 40 and a first rotating member also referred to herein as a reversible cam 50 generate the characteristic reciprocating drive for operation of the needle 10. Generally, the swing arm 30 is a linking member which provides the mechanical connection between the driving elements and the tattoo elements. While generally referred to as a swing arm, it may include multiple elements or may be arranged in a different way to accomplish the same thing, provide the interconnection between the driving elements and the tattooing elements. The driving elements generally refers to the drive element 22 which converts the rotary motion of the motor to a linear motion for operation of the swing arm 30. In addition, the swing arm 30 and/or the follower arm 32 may provide dampening structure to absorb any reactionary forces from operation of the tattoo needle 10.

The biasing member 36 generally allows for more consistent compression allowing for smoother operation as the cam 50 rotates. As depicted, the biasing member 36 provides resistance against the upward movement of the follower arm 32 and swing arm 30 during rotation of the cam 50. A rotational axis and an offset rotational axis extend through the protuberance 53, the rotational axis being angularly offset from the offset rotational axis. Generally, the offset rotational axis extends outwardly from the motor shaft 41 and the rotational axis extends centrally through the cam 50 as the protuberance 53 is rotated. The motor shaft 41 is received by the offset aperture 53b and generally rotates the rotational axis aligned with the cam 50 about the offset rotational axis.

As illustrated in FIG. 3, the offset aperture 53b is spaced from the rotational axis, also referred to as a central axis which extends centrally through the central aperture 53a and cam 50. Offset rotation occurs when the motor 40 rotates the cam 50 in a circular path, radially spaced from the offset axis which extends through the offset aperture 53b and in the depicted embodiment is spaced from the central axis.

During the offset rotation, the biasing member 36 allows for compression of the follower arm 32 and provides a reactionary bias against movement of the follower arm 32 during the offset rotation. In this way, the biasing member 36 damps or otherwise limits upward movement of the swing arm 30. Cam 50 includes an outer surface with a first surface (also referred to as a first flank) and a second surface (also referred to as a second flank) separated by a nose 54 generally characterized as a raised region or bump along the outer surface of the cam 50. Generally, the first surface and second surface are non-symmetrical. During rotation of the cam 50 upon engagement with the swing arm bearing 34 an oscillating motion is generated. For example, during operation of the motor 40 the nose 54 periodically rotates towards the swing arm bearing 34. As the nose 54 engages the swing arm bearing 34 it presents a downwardly directed motion. As a result of the downwardly directed motion on the swing arm bearing 34, the biasing member 36 provides a reactionary upward force between the follower arm 32 and the swing arm 30. This oscillating characterized by the upward and downward reciprocating motion is transferred to the swing arm 30. The biasing member 36 encourages the swing arm 30 to return to the top of its pivoted operation during use, and particularly after the motor 40 has stopped. The biasing member 36 also helps to maintain mechanical contact between the swing arm 30 and the cam bearing 48. In this way, the biasing member 36 provides for more consistent compression and smoother operation of the machine 20.

As further depicted in FIG. 3, the shaft of motor 40 is threadably received by cam base 44 which includes connecting structure for connection to the cam also referred to as a cam lobe 50. A cam bearing 48 is received between cam 50 and the cam base 44 for rotational engagement with the follower arm 32 during rotation. An exemplary fastener 52 secures the cam 50 to the cam base 44. As illustrated, the cam 50 is offset from the rotational axis of the motor, which runs through the shaft 41.

The illustrative cam 50 includes a nose 54 which is pointed and opposite heel 56. The nose 54 separates a first flank 58 from a second flank 59, the first flank 58 being generally flat while the second flank 59 has a generally arcuate surface. The central section of the cam 50 includes a protuberance 53 which extends centrally outwardly for connection to the cam base 44 and for supporting the cam bearing 48. The protuberance 53 depicted in FIG. 3 includes an offset aperture 53b which receives shaft 41 and is generally offset from the central aperture 53a which receives fastener 52. In general, the cam 50 translates the rotary movement of the motor 40 to pivoted rotational movement of the swing arm 30 and thus to reciprocating motion to the needle 10 like a traditional stroke wheel. The cam 50 may have an alternatively configured exterior surface having varying properties which can be used to control the depth and stroke length of the needle 10 or the speed or strength of the needle 10.

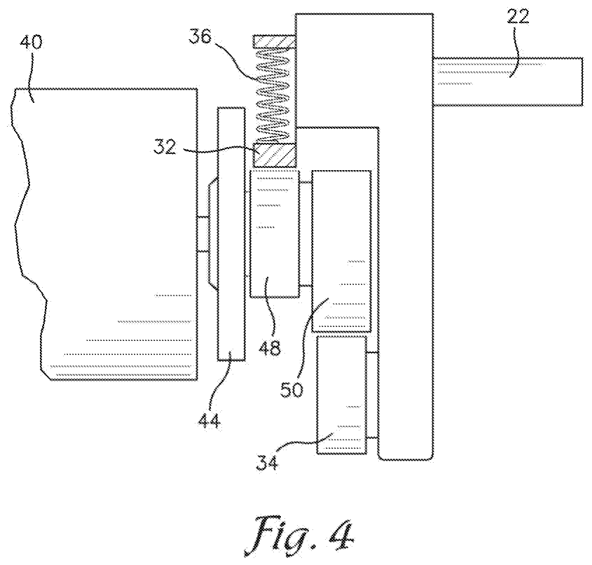

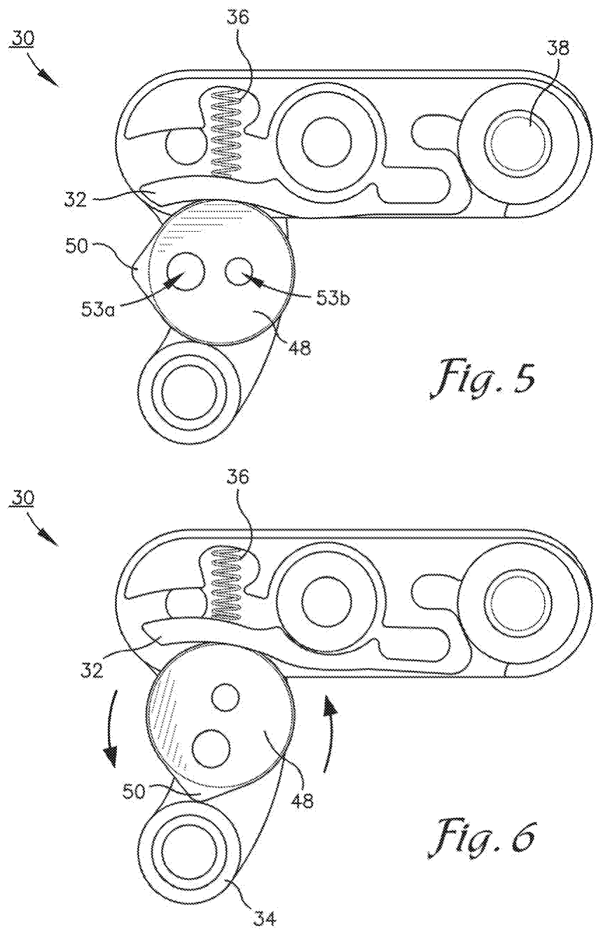

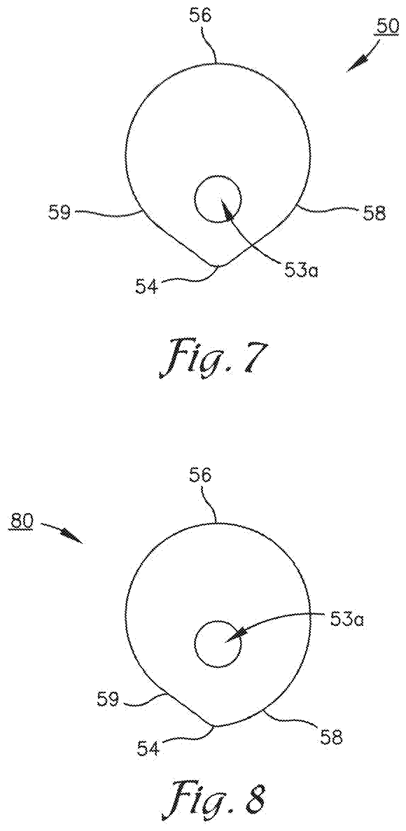

As further depicted in FIGS. 4-5, during operation, the motor 40 is energized by connecting terminals 42 to an electric power source. An externally adjustable power source and optional foot pedal (not shown) may be used as desired. Depending on the polarity of the terminals, the motor 40 will rotate clockwise or counterclockwise. During clockwise rotation, the motor 40 rotates cam base 44 which rotates cam 50 about the offset aperture 53b. During rotation of the cam base 44 and cam 50, the cam bearing 48 rotates in engagement with the following arm 32. As the surface of the cam bearing 48 rotates about the offset aperture 53b, the follower arm 32 is biased against the outer surface of the cam bearing 48 while allowing for compression and decompression of the follower arm 32. Generally, the cam bearing 48 has a smooth metallic surface and is depicted as circular although other friction reducing surfaces and shapes may be utilized for engagement between the follower arm 32 and the cam bearing 48.

During rotation of the cam 50, the offset aperture 53b rotates and the cam 50 engages the swing arm bearing 34, causing the swing arm 30 to rotate angularly upward. More specifically, when the nose 54 of the cam 50 rotates downward, the nose 54 engages the swing arm bearing 34 and forces the swing arm 30 upward. Depending on the rotation of the motor 40, as the first flank 58 engages the swing arm bearing 34, the swing arm 30 accelerates downward driving the drive element 22 and the needle 10 down, providing a harder needle 10 hit. As the nose 54 engages the swing arm bearing 34, the direction of the needle 10 is reversed and engagement between the swing arm bearing 34 and the second flank 59 slows the needle 10, providing a softer needle 10 hit. In this way, the operator can alter the needle hit characteristics by, for example, providing a soft or hard hit of the needle 10 simply by reversing the polarity of the terminals 42, as desired.

The housing 24 retains and supports the constituents of the present invention while providing a motor-receiving tube 35 and a tower housing 36. The tower housing 36 is adjacently and perpendicularly connected to the motor-receiving tube 35, similar to traditional tattoo machine designs.

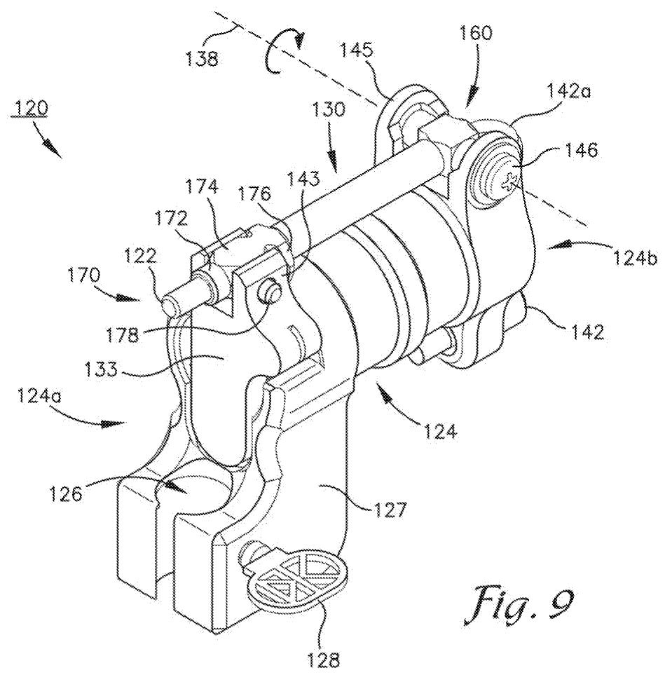

A second embodiment of the of the reversible rotary tattoo machine 120 is illustrated in FIG. 9. The tattoo machine 120 includes an alternative housing 124 with a cylindrical passage 125 adapted for receipt of the generally cylindrical reversible motor 40, and a central armature canal 126 adapted for reciprocal receipt of the armature (depicted in FIG. 1 as reference 12), the cylindrical passage 125 being generally perpendicular to the central armature canal 126.

An alternative swing arm 130 is depicted in FIG. 9. Generally, the alternative swing arm 130 may include any mechanical linkage and may be divided into multiple components or one component to generally connect the driving elements to the tattooing elements, the driving element 122 generally converting the rotary motion of the motor 40 to a linear motion which is translated to the mechanical linking members or alternative swing arm 130. In FIG. 9, the alternative swing arm 130 is depicted as extending rearwardly from a front housing portion 124a, along the alternative housing 124 towards a rear housing portion 124b. As depicted, the front housing portion 124a is generally located near the central armature canal 126 and the rear housing portion 124b is located near a terminal 142. Generally, the alternative swing arm 130 depicted in FIG. 9 has a generally cylindrical body which includes a proximate cylindrical passage 146 and a distal cylindrical passage 146. Generally, the proximate and distal cylindrical passages 146, 147 extend through the cylindrical body. The distal cylindrical passage 147 is generally associated with a distal end of the alternative swing arm 130 and the proximate cylindrical passage 146 is generally associated with a proximate end of the alternative swing arm 130.

Generally, the proximate cylindrical passage 146 and distal cylindrical passage 147 are adapted for mounting the swing arm 130 along the alternative housing 124. More specifically, the proximate cylindrical passage 146 is adapted for mounting the alternative swing arm 130 to the radial guide 133. The distal cylindrical passage 147 is adapted for mounting the alternative swing arm 130 along a pivot axis 138 for pivotal operation of the swing arm 130 about an upending mounting bracket 145.

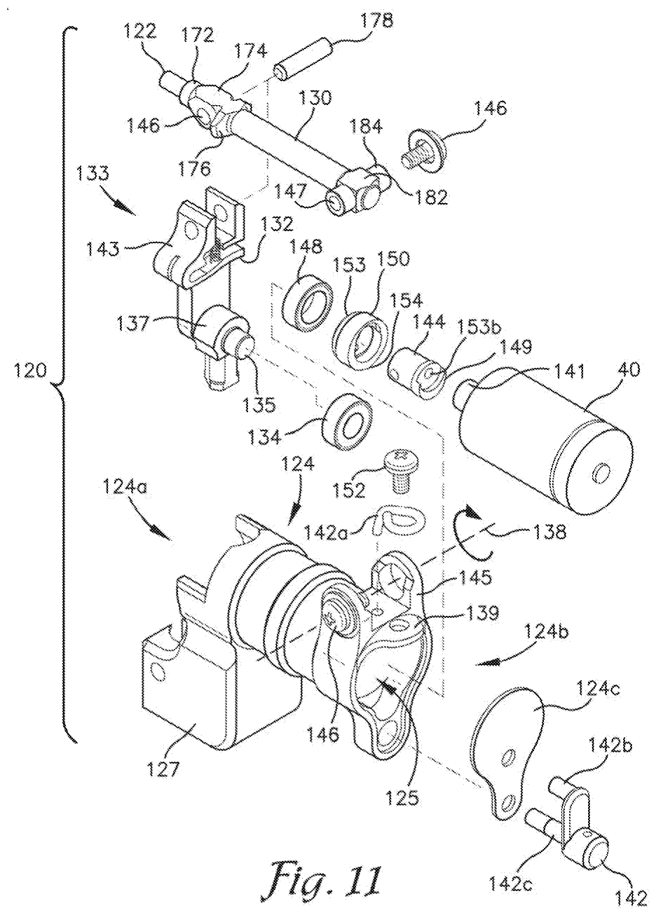

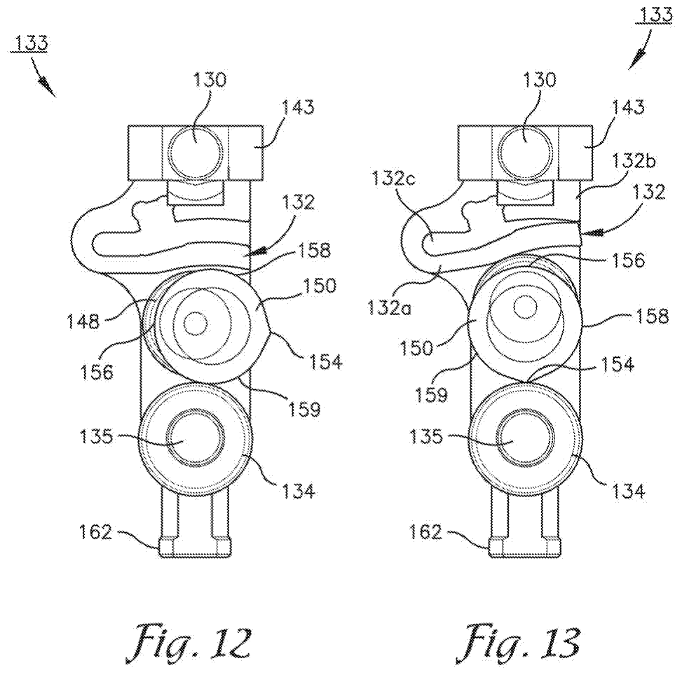

The alternative swing arm 130 includes an alternative drive element 122 for operation of a tattoo needle (depicted in FIG. 1 as reference 10). The alternative drive element 122 depicted in FIG. 9 is in reciprocal communication with an armature (depicted in FIG. 1 as reference 12). In operation, the alternative drive element 122 exhibits generally reciprocating linear motion which is converted from the rotary motion of the reversible motor 40 through bearings and mechanical linkage members which are then connected to the alternative swing arm 130 which includes the alternative drive element 122. In operation the reversible motor 40 is operated in either a clockwise or counter clockwise direction by alternating the polarity of the power source (not shown) connected to the first and second electrical connectors 142a and 142b. Upon rotation of the reversible motor 40, the motor shaft 141 rotates a first rotating member like the alternative cam base 144 which presents an asymmetrical outer surface for rotation of a second rotating member, like the radial guide bearing 134. Generally, the asymmetrical outer surface presented by the alternative cam base 144 is depicted as alternative cam 150 in FIG. 11. As further illustrated in FIGS. 12, 13, the asymmetrical outer surface of alternative cam 150 generally includes a first surface 158 separated from a second surface 159 by a bump 154 which is opposite a heel 156, the first surface 158 and second surface 159 being non-symmetrical, the heel 156 being generally curved and with a shorter radius than the bump 154. As depicted in FIG. 12, 13, the bump or nose 154 extends outwardly from the center of the alternative cam 150. The second rotating member depicted as the radial guide bearing 134 is generally circular. Alternatively, the alternative cam 150 and radial guide bearing 134 may have varying configurations different from those depicted and may be more curved, less curved, shorter or longer, more tapered or less tapered as desired for operation in the reversible rotary tattoo machine 120.

Generally, the embodiment of the reversible rotary tattoo machine 120 depicted in FIG. 9 converts the rotation of the reversible motor 40 to a linear reciprocating motion which is presented by the alternative drive element 122 during linear operation of the armature 12. As the alternative drive element 122 moves up and down, the armature 12 passes through the central armature canal 126, reciprocating the needle 10. Generally, the central armature canal 126 extends through a lower boss 127 and may optionally include a thumbscrew 128 extending therethrough for adjustment of the central armature canal 126.

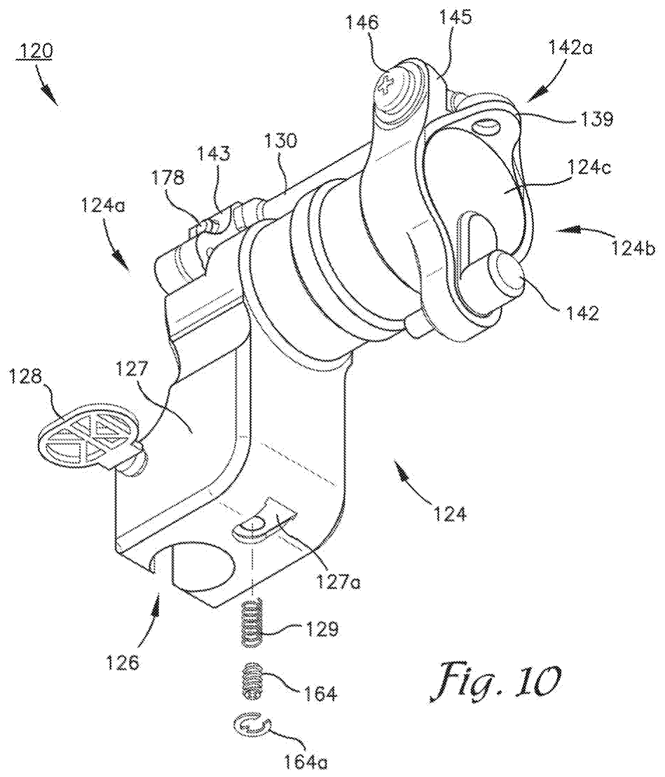

Generally, the alternative housing 124 includes a cylindrical opening 125 for receipt of the reversible motor 40. The alternative swing arm 130, as a mechanical linking member, extends in an overlying position along the alternative housing 124 between a pair of mounting brackets (143, 145). During operation of the reversible motor 40, the alternative swing arm 130 depicted in FIGS. 9-10 pivots up and down as the reversible motor 40 rotates. The cylindrical opening or passage 125 depicted in FIG. 11 extends from a proximal cylindrical passage 146 to a distal cylindrical passage 147, the proximal and distal cylindrical passages 146, 147 being in open communication with each other. In the depicted embodiment, the proximal cylindrical passage 146 is angularly oriented to and adjoins the lower boss 127.

The proximate end 170 of the alternative swing arm 130 includes the outwardly extending alternative drive element 122 adapted for engagement with the armature 12, the proximate end 170 having an outer collar 172 which provides sufficient structure for vertical alignment of the armature 12 with the central armature canal 126.

A junction 174 depicted in FIG. 9 and referred to herein as a flattened junction, extends between the outer collar 172 and a bracket spacer 176. The junction 174 presents the proximate cylindrical passage 146 and is configured for receipt by the radial mounting bracket 143 presented by the radial guide 133. Generally, the radial mounting bracket 143 extends upwardly from the radial guide 133 for receipt of the proximate end 170 at junction 174. The proximate cylindrical passage 146 is generally adapted for removably receipt of the radial pin 174 which secures the junction 174 to the radial mounting bracket 143.

The bracket spacer 176 depicted in FIG. 9 provides a lip or alignment structure for engagement by the proximate end 170 of the alternative swing arm 130 with the radial mounting bracket 143. The bracket spacer 176 generally assists in alignment of the proximate end 170 of alternative swing arm 130 along the top of the alternative housing 124. In addition, passage of the radial pin 174 through the proximate cylindrical passage 146 at junction 174 and the radial mounting bracket 143 maintains alignment of the proximate end 170 of the alternative swing arm 130 during pivoted operation.

The distal end 180 of the alternative swing arm 130 is depicted in FIG. 9 as being spaced opposite the proximate end 170. Generally, the distal end 180 includes a rear junction 182 presenting a cylindrical conduit 184 with a pair of openings, each of which is adapted for receipt of a pair of rotatable fasteners 146. Generally, the cylindrical conduit 184 is adapted for receipt of the rotatable fasteners 146 and for pivoted operation of the alternative swing arm 130 about a pivot axis 138. Generally, the pivot axis 138 extends between the cylindrical conduit 184 and the rotatable fasteners 146 which secure the cylindrical conduit 184 through the upending mounting bracket 145 for pivoted rotation. The cylindrical conduit 184 may or may not be threaded, but generally, has sufficient dimensions for receiving the pair of rotatable fasteners 146 extending through the open sides of the upending mounting bracket 145. The pivot axis 139 generally corresponds to axis about which the alternative swing arm 130 pivots.

The rear junction 182 has generally complementary configuration for receipt by the upending mounting bracket 145 while allowing for pivoted rotation of the alternative swing arm 130. A first electrical connector 142a is secured to a flange 139 which extends rearwardly from the rear housing portion 124b associated with the alternative housing 124. As depicted in FIG. 10, the flange 139 presents a receiver for mechanical receipt of a flange fastener 152 in electrical contact with the first electrical connector 142a.

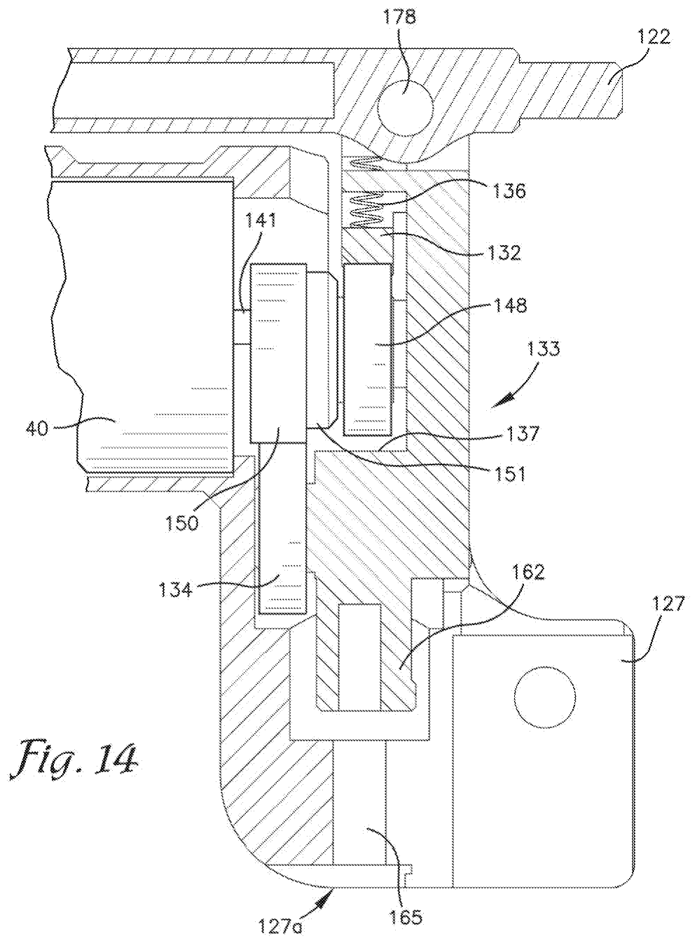

FIG. 10 illustrates the lower boss 127 with a contoured recess 127a also referred to as a vertical support receiver. The contoured recess 127a presents a channel 165 (illustrated in FIG. 14) which may be threaded and extends inwardly towards the radial guide 133 and is adapted for receipt of an elongated fastener 164 which allows for adjustment or alignment of the radial guide 133. Additionally, a biasing member 129 may be utilized to provide flexibility in the adjustment or alignment of the radial guide 133. The elongated fastener 164 is mechanically secured within the contoured recess 127a with a retainer 164a. In the depicted embodiment, the retainer 164a is configured as a standard e-clip but any standard or non-standard retainer which provides the desired functionality could be utilized. One end of the elongated fastener 164 may include a shaped tip, such as but not limited to a hexagonal tip for engagement by a standard hexagonal tool. As the shaped tip is rotated the elongated fastener rotates for threaded adjustment of the elongated fastener 164 within the channel 165 as desired. In this way, the positioning or alignment of the radial guide 133 can be tuned or aligned as desired.

In operation, the biasing member 129 may be “backed-off” by rotating the elongated fastener 164, while retaining the biasing member 129 within the alternative housing 124. The radial guide 133 may be tuned or adjusted by adjusting the pressure exerted upon the biasing member 129 by the elongated fastener 164 to provide the desired movement of the alternative swing arm 130 which a user may desire. The range of adjustment may vary based upon the tension and position of the biasing member 129 in relation to the elongated fastener 164. By providing for threaded adjustment of the biasing member 129, a user may easily adjust the operational characteristics of the reversible rotary tattoo machine 120. Alternatively, a different biasing member may be inserted through the channel 165 by removing the retainer 164a and the elongated fastener 164 to easily and simply provide different operational characteristics.

For example, while a particular pressure on the needle 10 may be necessary for use of the reversible rotary tattoo machine in coloring, a different pressure on the needle 10 may be desired for doing detailed work such as fine lines. By providing ready access to the channel 165 through the lower boss 127, the pressure exerted upon the radial guide 133 by the biasing member 129 may be quickly and easily adjusted or changed without disassembling the entire machine. Generally, the tension for the biasing member 129 may range from about 0.5 to 3.5 lbs/in.

As further depicted in FIGS. 11, 14 and 15, the reversible motor 40 includes alternative motor shaft 141 which is generally adapted for rotational engagement with an alternative cam base 144 providing offset angular rotation to the alternative cam 150. As depicted in FIG. 11, the alternative cam base 144 includes an annular protuberance 153 having an outer circumference corresponding to an inner surface of the alternative cam bearing 148, the outer circumference being adapted for engagement between the annular protuberance 153 and the alternative cam bearing 148. Generally, the alternative cam bearing 148 receives the annular protuberance 153. In an alternative embodiment, the alternative cam base 144 may be manufactured or otherwise combined with the alternative cam 150 as a unitary piece to assist in the manufacturing and assembling process.

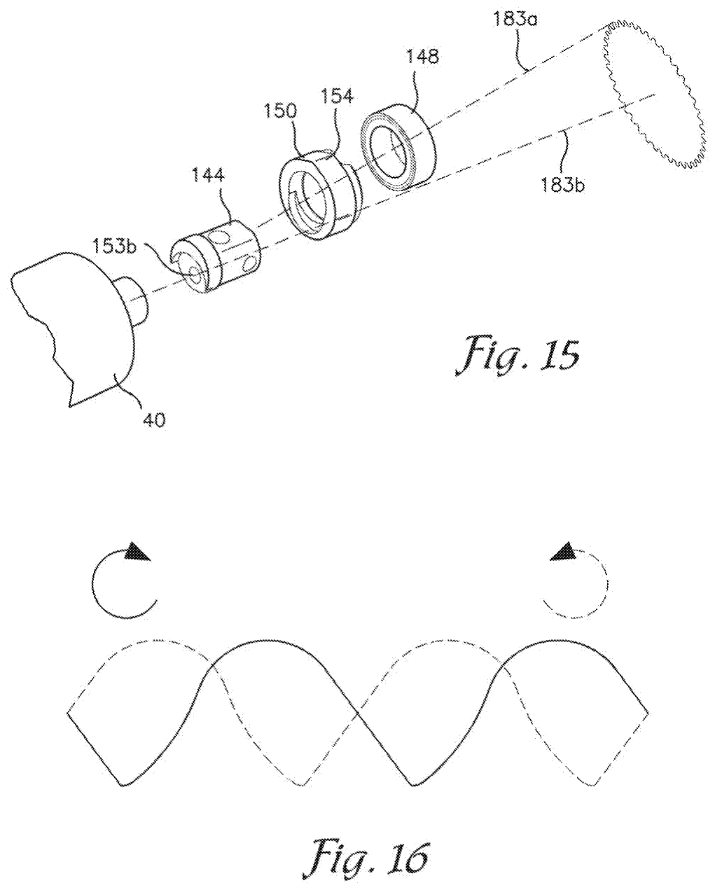

As depicted in FIG. 15, an offset rotational axis 183a is angularly spaced from the rotational axis 183b extending through the motor shaft 141. The annular protuberance 153 includes an alternative offset aperture 153b which is aligned with the alternative motor shaft 141 by the arcuate spacer 149. The annular protuberance 153 also includes an alternative central aperture 153a which extends centrally through the annular protuberance towards the alternative cam bearing 148. The alternative offset aperture 153b and the alternative central aperture 153a are spaced radially apart, with the alternative central aperture 153a having a greater radius than the alternative offset aperture 153b.

As the reversible motor 40 rotates the motor shaft 141, the rotational axis 183b extends outwardly from the alternative motor shaft 141 through the offset aperture 153b and the offset rotational axis 183a extends outwardly from the alternative central aperture 153a centrally through the alternative cam bearing 148. During rotational operation of the reversible motor 40, the offset aperture 153b is rotated in sync with the central aperture 153a. The offset rotational axis 183a extends from the central aperture 153a towards the bearing support 135 associated with the radial guide 133. The arcuate spacer 149 aligns the motor shaft 141 with the offset aperture 153b.

As generally depicted in FIGS. 12-13, the alternative cam 150 has an outer surface with a first surface 158 and a second surface 159 separated by a bump 154. As the alternative cam 150 rotates its outer surface presents varying characteristics to the engaged radial guide 133 which are translated to the alternative drive 122 for operating the armature 12. As depicted in FIG. 15-16, the rotation of the alternative cam 150 upon the radial guide bearing 134 corresponds to a first periodic movement, which may be generally referred to as a nutation or slight back and forth deviation. Rotation of the alternative cam 148 in communication with the reversible motor 40 by the alternative cam base 144, rotates the offset rotational axis 183a about the rotational axis 183b corresponds to a second periodic movement which may be generally described as a precession about the rotational axis 183b.

Generally, the alternative cam 150 outer surface provides a cyclical rotation to radial guide 133 each time the alternative cam 150 engages the radial guide bearing 134 as the outer surfaces of each engage each other during operation of the motor 40. During operation, the alternative cam bearing 148 is positioned for engagement with an alternative follower arm 132 during rotational contact between the alternative cam 150 and an alternative radial guide bearing 134.

In general, the alternative follower arm 132 stabilizes the radial guide 133 during operation while at least partially damping the movement of the radial guide 133. As depicted in FIG. 11, the alternative follower arm 132 is in mechanical communication but separate from the alternative swing arm 130. The alternative follower arm 132 includes a lower arm 132a spaced from an upper arm 132b, the lower and upper arm 132a, 132b surrounding an interstice 132c and in communication with each other with biasing member 136. The interstice 132c provides sufficient separation between the lower and upper arm to allow for deflection of the lower arm 132a during operation of the radial guide 133. Generally, the biasing member 136 provides a reactionary force between the lower and upper arm 132a, 132b.

The alternative cam base 144 has a generally axially symmetric exterior with an arcuate spacer 149 which rotationally offsets the rotational axis of the motor shaft 141 from the central rotational axis extending through the alternative cam 150. The rotational axis of the motor shaft 141 is referred to herein as the rotational axis 183b and the central rotational axis extending through the alternative cam 150 is referred to as the offset rotational axis 183a. The distance between the rotational axis 183b and the offset rotational axis 183a at the alternative cam base 144 may be generally between 0.03 and 0.1 and more specifically, may be about 0.075.

As further depicted in FIG. 11, the radial guide 133 includes a bearing support 135 spaced from the radial mounting bracket 143. The alternative follower arm 132 is generally positioned between the bearing support 135 and the radial mounting bracket 143 for selective engagement by the cam bearing 148 during operation of the reversible motor 40.

Generally, the radial guide 133 translates the first and second periodic motions to linear motion for pivoting the alternative swing arm 130 between an upward and downward position as desired. The bearing support 135 generally receives the radial guide bearing 134 and allows for rotation. A shoulder 137 is raised and offset from the bearing support 135 for maintaining offset alignment of the radial guide bearing 134 on the bearing support 135 while the alternative cam bearing 148 is positioned inwardly from the radial guide bearing 134 and vertically supported above between the shoulder 137 and the alternative follower arm 132. While, the shoulder 137 is depicted with a partially arcuate surface, it is not limited to such a configuration and may include a number of configurations as would be understood by one skilled in the art.

The rear housing portion 124b is depicted in FIGS. 10 and 11 with a housing cover 124c which encloses and protects the reversible motor 40 during operation from ambient conditions or undesired electrical contact. The illustrated embodiment of the housing cover 124c has a teardrop configuration, although other configurations may be utilized. Generally, the housing cover 124c is positioned between the reversible motor 40 and the terminal 142 to help insulate the reversible motor 40.

The terminal 142 is located on one end of the alternative housing 124 and in electrical communication with the reversible motor 40. Generally, the terminal 142 presents the second electrical connector 142b. Depending on the polarity of the connected power source (not shown), one of the first and second electrical connectors 142a, 142b is associated with a positive terminal and the other of the first and second electrical connectors 142a, 142b is associated with a negative terminal. In general, the first electrical connector 142a includes a downwardly depending leg extending from a circular opening. The circular opening associated with the first electrical connector 142a receives flange fastener 152 which passes through the circular opening into the receiver located on the flange 139. The downwardly depending leg extends through the alternative housing 124 into the cylindrical passage 125, securing the reversible motor 40. Generally, the first electrical connector 142a is in electrical communication with the alternative housing 124 and the second electrical connector 142b is in electrical communication with the reversible motor 40.

FIGS. 12 and 13 illustrate the radial guide 133 in the upward position in FIG. 12 and the downward position in FIG. 13. As illustrated, the alternative swing arm 130 extends through the radial mounting bracket 143. During rotational operation, as the alternative cam 150 rotates, the bump 154 rotates towards the radial guide bearing 134. Upon engagement of the bump 154 by the radial guide bearing, the alternative follower arm 132 compresses inwardly and the radial guide bearing 134 is directed downward presenting a downward thrust to the radial guide 133. In addition, when the alternative follower arm 132 compresses, the biasing member 36 compresses and provides a reactionary force.

A vertical support 162 is illustrated in FIGS. 12 and 13, the vertical support 162 being adapted for receipt within the channel 165 and accessible through the vertical support receiver 127a located on the lower boss 127. The vertical support 162 also includes a pair of grooves which are received by the channel 165. In addition, an elongated fastener 164 may be used to secure the vertical support 162 during operation. The vertical support receiver 127a generally provides access for adjustment of the radial guide 133 from the lower boss 127 during operation.

The alternative embodiment illustrated in FIGS. 12-14, provide for upward and downward pivoted movement of the alternative swing arm 130 during operation of the motor 40 which rotates the motor shaft 141. The alternative following arm 132 is spaced from the alternative swing arm 130 with biasing member 36 which allows for inward compression and outward decompression of the alternative following arm 132 during rotational operation of the motor 40.

The alternative swing arm 130 in conjunction with the reversible motor 40 and the alternative cam 150, which are reversible, generates the characteristic reciprocating drive for reciprocal operation of the alternative reversible tattoo machine 120.

As previously stated, the biasing member 36 generally allows for more consistent compression for smoother operation as the alternative cam 150 rotates. As depicted, the biasing member 36 reacts to any upward movement of the alternative follower arm 132 during rotation of the alternative cam 150 by the reversible motor 40.

The arcuate spacer 149 is contoured for receipt of the motor shaft 141 and alignment of the shaft 141 with the offset aperture 153b. As the motor shaft 141 rotates, the alternative cam base 144 rotates along an elongated path, also referred to as precession, about the axis of rotation 183b associated with the offset aperture 153b. The alternative motor shaft 141 is received by the arcuate spacer 149 for rotating the alternative cam 150.

As illustrated in FIG. 14, the alternative motor shaft 141 is radially offset from the center of the alternative cam bearing 148. As the reversible motor 40 is operated, the alternative cam 150 rotates about the offset rotational axis 183a. In operation of the reversible motor 40, the offset rotation presents unique downward and upward thrust characteristics for the alternative swing arm 130.

During the offset rotation, the biasing member 36 allows for compression of the alternative follower arm 132 and provides bias against upward movement of the alternative follower arm 132. In this way, the biasing member 36 limits upward movement of the alternative swing arm 130 for engagement of the bump 154 upon the radial guide bearing 134. The biasing member 36 also directs the alternative swing arm 130 towards a home position associated with the upward position of the alternative swing arm 130 during use, and particularly after the reversible motor 40 has stopped. The biasing member 36 also helps to keep the alternative swing arm 130 in mechanical contact with the alternative cam bearing 148. In this way, the biasing member 36 provides for more consistent compression and smoother operation of the machine 20.

During operation of the motor 40, the drive element 122 associated with the swing arm proximate end 170 pivots up and down in a non-uniform manner which corresponds to two periodic motions generated by the embodiment of the reversible rotary tattoo machine 120. As the alternative swing arm proximate end 170 thrusts up and down, the swing arm distal end 160 maintains a generally stationary position with respect to the pivot axis 138 with, for example, mechanical fasteners 146. The pivotal axis 139 generally extends through the distal cylindrical passage associated with the cylindrical conduit 184 extending between the upending mounting bracket 145.

As indicated in FIG. 15 the first periodic movement is illustrated as a generally periodic back and forth oscillation while the second periodic movement is illustrated as a generally elongated or circular path precession which oscillates as a result of the first periodic movement.

The first periodic movement is depicted in FIG. 16 as a generally sinusoidal wave, where at least a portion of the movement is linear and the speed at the downward portion of the cycle is accelerated such that the needle 10 has a shorter duration at the bottom of the cycle and its speed is quicker allowing it to move upward quickly from the downward portion of the cycle. The first periodic movement is generated as the first rotating member, the alternative cam 150, rotates upon the second rotating member, the radial guide bearing 134. The outer surface of the alternative cam 150 presents a non-uniform surface which effects the characteristics of the first periodic movement. The first periodic movement is also effects as the polarity of the reversible motor is reversed, for example, from a clockwise direction to a counter-clockwise direction where the first periodic movement in a clockwise direction is different from the first periodic movement in a counter-clockwise direction.

The second periodic movement is generated by the reversible motor 40 the rotation of the offset rotational axis 183a about the rotational axis 183b, the offset rotational axis extending centrally through the alternative cam base 144, the alternative cam 150 and the alternative cam bearing 148.

In operation, the alternative motor shaft 141 is aligned with the offset aperture 153b by the arcuate spacer 149. As further illustrated in the exploded view of FIG. 15, the rotational axis 183b extends from the alternative motor shaft 141 through the offset aperture 153b. The offset rotational axis 183a extends from the alternative cam base 144 centrally through the alternative cam 150 (which in operation circumscribes the alternative cam base 144) and the alternative cam bearing 148 (which in operation adjoins the alternative cam base 144). The second periodic movement is generated when the reversible motor 40 rotates the alternative motor shaft 141, rotating the alternative cam base 144 along an elongated path with the offset rotational axis 183a encircling the rotational axis 183b as depicted in FIG. 15.

Specifically, the first periodic movement is provided by the torsion generated by the rotation of the alternative cam base 144 in relation to the alternative motor shaft 141 positioned by the arcuate spacer 149 such that rotational torques in the cam base 144 are related but differential to the transverse angular velocity of the alternative cam 150 upon the radial bearing 134. These torques tend to rotate the alternative cam 150 and alternative cam bearing 148 together, separated from the rotational axis of the motor shaft 141. The differential provides a proportional motion along two paths, one corresponding to the displacement of the offset rotational axis 183a from the rotational axis 183b and the other related to the motion exhibited by the offset rotational axis 183 as a result of the engagement of the alternative cam 150 upon the radial guide bearing 134 which is transmitted to the radial guide 133.

The exterior surface of the alternative cam 150 is generally asymmetrical with first surface 158 having a curvature different from the second surface 159, the first surface 158 being separated from the second surface 159 by the bump 154 on one side and the heel 156 on the other side. Generally, the first surface 158 in the depicted embodiment of FIG. 11 is less curved than the second surface 159. In the depicted embodiment, the greater curvature corresponds to a slower velocity and less curvature corresponds to a faster velocity.

In FIG. 16 illustrates this oscillation which varies based on the rotation of the reversible motor 40 which varies based on the polarity of the first and second electrical connectors 142a, 142b. At a resting position, the alternative cam 150 engages the radial guide bearing 134 at a heel 156, for example. Generally, the heel 156 is located on the outer surface of the alternative cam 150 opposite the bump 154. As the alternative cam 150 is rotated clockwise, the oscillation begins from the resting position at a crest which corresponds to a minimum displacement of the offset rotational axis 183a from the rotational axis 183b. As the alternative cam 150 is rotated clockwise, the displacement increases along a generally linear downward slope as the radial guide bearing 134 moves along the first surface 158. Upon contact between the radial guide bearing 134 with the bump 154, the oscillation corresponds to a peak displacement between the offset rotational axis 183a and the rotational axis 183b. A sharp change is presented as the bump 154 comes into contact with the radial guide bearing 134. Continued rotation of the alternative cam clockwise 150 moves the radial guide bearing 134 along the second surface 159 with a slightly arcuate approach as the offset rotational axis 183a comes closer to the rotational axis 183a until the radial guide bearing 134 comes into contact with the heel 156. At the resting position, the offset rotational axis 183a comes closest to the rotational axis 183b while the offset rotational axis 183a precesses about the rotational axis 183b during the rotational operation of the reversible motor 40. In counter-clockwise rotation the oscillation will reverse as illustrated by the dashed line in FIG. 16.

The upward or downward thrust of the armature (not shown) is then a linear function of the oscillation of the offset rotational axis 183a as it precesses along the rotational axis 183b during operation of the reversible motor 40.

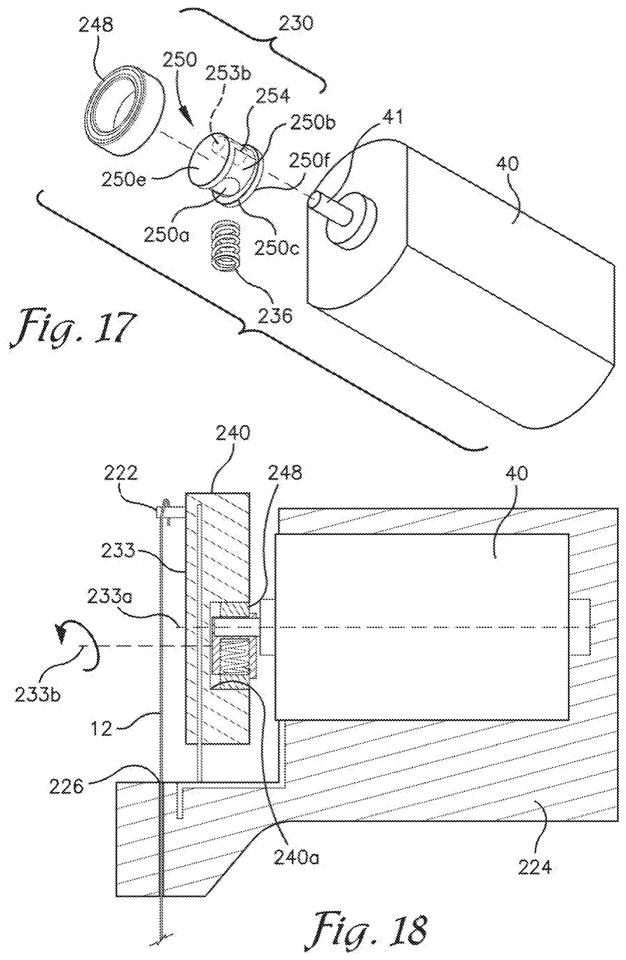

FIG. 17 illustrates a centric cam assembly 230 in an exploded orientation with a centric cam 250 having a cylindrical structure adapted for receipt by a centric cam bearing 248. The centric cam 250 includes a cylindrical receiver 250b for receiving the centric cam bearing 24. The centric cam 250 also includes a side port 250a for receipt of the biasing member 236 and an offset aperture 253b for receipt of the motor shaft 41.

The centric cam assembly 230 illustrated in FIGS. 17-19 includes an offset rotational axis 233a spaced from a rotational axis 233b. The offset rotational axis 233a extends from the motor shaft 41 while the rotational axis 233b is centered about the centric cam 250. As the reversible motor 40 is operated, the centric cam 250 rotates about the offset rotational axis 233a.

Actuator 240 is operationally connected between the motor 40 and the armature 12 and provides the desired reciprocal movement for tattooing. During operation, the centric cam assembly 230 rotates about the motor shaft 41 in an offset manner, rotating the engaged centric cam bearing 248. The centric cam bearing 248 is received within a recess 240a of the actuator 240 for reciprocal engagement.

As the centric cam bearing 248 rotates, the actuator 240 reciprocates driving the armature 12 repeatedly from an upper to a lower position. The rotation of the motor shaft 41 rotates the centric cam 250 about the motor shaft, allowing for offset rotation of the centric cam bearing 248.

Receipt of the biasing member 236 within the side port 250a provides a counter force which dampens the offset rotation of the centric cam bearing 248 while allowing for substantially full rotation of the centric cam bearing 248. During rotation of the centric cam 250 the biasing member 236 presses between the centric cam bearing 248 and the centric cam 250. As the centric cam 250 rotates, the biasing member 236 exerts an outward force upon the centric cam bearing 248 which causes the rotation to quickly decelerate. When the centric cam 250 stops rotating, linear movement of the armature 12 stops and the interconnected needle (not shown) reverses direction and is retracted.

The centric cam assembly 230 generally allows for smoother operation during rotation of the centric cam 250. As depicted, the biasing member 236 provides internal bias against the centric cam bearing 248 during rotational of the centric cam 250 received by the centric cam bearing 32 and actuator 240 during rotation of the centric cam 250.

Generally, the rotational axis 233a and offset rotational axis 233b extend from the motor 40 through the centric cam 250, the rotational axis 233a being angularly offset from the offset rotational axis 233b. Generally, the offset rotational axis 233b extends outwardly from the motor shaft 41 and the rotational axis extends centrally through the centric cam 250 and the centric cam bearing 248 as the motor 40 is operated. The motor shaft 41 is received by the second alternative offset aperture 253b and generally rotates the centric cam 250 in alignment with the centric cam bearing 248 about the offset rotational axis 233a.

As illustrated in FIG. 18, the offset aperture 253b is spaced from the rotational axis 233b, which extends centrally along the centric cam 250 and the centric cam bearing 248. Offset rotation occurs when the motor 40 rotates the centric cam 250 in a circular path about the offset axis 233a which extends through the offset aperture 253b and in the depicted embodiment is radially spaced from the rotational axis 233b.

During the offset rotation, the biasing member 236 provides rotational interference during rotation of the centric cam 250 within the centric cam bearing 248 with an internal counter-revolutionary force which provides resistance to offset rotation. In this way, the biasing member 236 dampens or otherwise limits upward or downward movement of the actuator 240.

The centric cam 250 includes an outer cylindrical surface 250d extending from a bottom 250e to a top 250f with a radial groove 250b and an outer sidewall support 254 extending therebetween. Generally, the radial groove 250b extends inwardly from the outer sidewall support 254 gradually along the outer cylindrical surface 250d. Generally, the outer sidewall support 254 separates the proximate and the distal end of the radial groove 250b. A spacer 250c having a varying radius extends around circumferentially along the bottom 250e, extending from a minimum associated with the outer sidewall support 254 to a maximum opposite the outer sidewall support 254. The outer sidewall support 254 presents a raised region or bump along the outer cylindrical surface 250d of the centric cam 250.

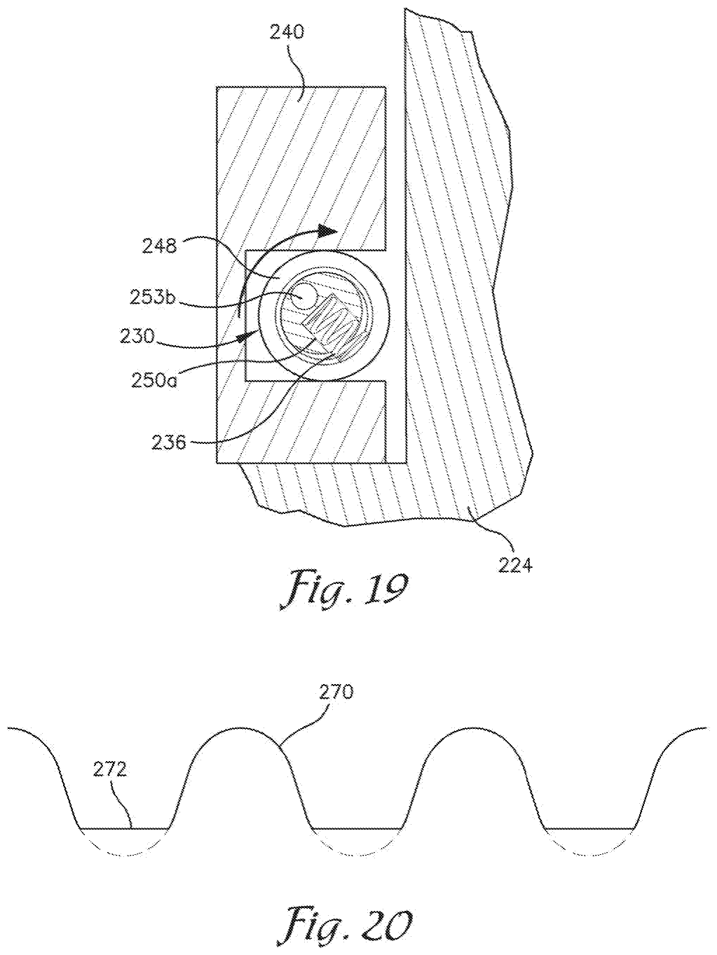

As further illustrated in FIG. 19, during rotation of the centric cam 250 upon engagement with the centric cam bearing 248 an oscillating motion is generated. For example, during operation of the motor 40 the outer sidewall support 254 periodically rotates upward and downward within the actuator 240 recess 240a. As the outer sidewall support 254 engages the actuator 240 an upward or downward directed motion is provided depending on which side of the recess 240a is engaged by the outer sidewall support 254. The armature 12 oscillates as a result of the motion of the actuator 254 by the centric cam assembly 230.

The biasing member 236 provides a reactionary force to mechanically alter the spacing of the centric cam assembly 230 within the recess 240a of the actuator 240, mechanically limiting rotation. The biasing member 236 provides mechanical contact between the centric cam bearing 248 and the centric cam 250. In this way, the biasing member 236 provides for more consistent compression and smoother operation of the machine 220.

During operation of the motor 40, the centric cam assembly 230 pivots the actuator 240 up and down in a non-uniform manner which corresponds to two periodic motions generated by the second embodiment of the reversible rotary tattoo machine 220. As the actuator 240 pivots up and down, the armature 12 thrust upward and downward accordingly.

A dampener 260 is illustrated in FIG. 19 and corresponds to the reaction of the biasing member 236 upon the centric cam bearing 248 during rotation of the centric cam 250. Generally, the dampener 260 presents a secondary motion upon the armature 12 during rotation of the centric cam assembly 230. As the centric cam assembly 230 is rotated about the offset axis 233b the biasing member 236 exerts a reactionary force upon the centric cam bearing 248 at the bottom of the rotation which allows for an abrupt impact on the rotational movement.

As indicated in FIG. 20 the first periodic movement of the actuator 240 is illustrated as a generally periodic up and down oscillation 270 which corresponds to a forward and reverse armature movement. A secondary movement of the actuator 240 which results from the dampener 260 is illustrated as a clip 272 (also referred to as a cut or stall) in the actuator's 240 oscillation 270. The clipped oscillation 272 results from the impact of the dampener 260 during rotation of the centric cam assembly 230. Generally, the dampener 260 is configured such that the clip 272 is applied at the bottom of the periodic oscillation 270 of the actuator 240. The movement of the dampener 260 is applied to the hit of the needle (not shown) during rotation of the centric cam assembly 230 to make an abrupt impact upon the downward stroke of the armature 12 in mechanical communication with the needle (not shown). The abrupt impact provides for a more effective piercing. In addition, the dampener 260 aids in a more responsive retraction associated with the reverse armature movement allowing for a more efficient and effective tattooing procedure.

Generally, the dampener 260 limits forward movement of the armature 12, resulting in a stall for a period of time which is a result of the speed of the rotation of the centric cam assembly 230 and the dimension of the dampener 260. The dampener 260 is generally located between the centric cam 250 and the centric cam bearing 248 and extending angularly therebetween as the biasing member 236 compresses back and forth. During rotation of the centric cam assembly 230, the dampener stalls the armature 12 as a result of the compression and elongation of the biasing member 236 between the centric cam 250 and the centric cam bearing 248. The periodic movement is depicted in FIG. 20 as a generally sinusoidal wave, where at least a portion of the movement is linear and the speed at the downward portion of the cycle is accelerated such that the armature 12 has a softer hit at the bottom of the cycle and its speed is accelerated quickly upon pick-up as the oscillations continue upon the conclusion of the cut-off.

It is to be understood that while certain forms of the present invention have been illustrated and described herein, it is not to be limited to the specific forms or arrangement of parts describer herein. Other arrangements or embodiments, changes and modifications not precisely set forth, which can be practiced under the teachings of the present invention are to be understood as being included within the scope of this invention as set forth in the claims below.

What is claimed and desired to be secured by Letters Patent:

1. An improved rotary tattoo machine with a reversible motor, said rotary tattoo machine comprising:
a housing;
a linking member secured to said housing;
a drive element received by said housing and in pivoted communication with said linking member; and
said drive element further comprises at least a first rotating member having an asymmetrical outer surface which oscillates said drive element during operation of said reversible motor.

2. The improved rotary tattoo machine of claim 1 wherein said first rotating member includes a first surface spaced from a second surface by a bump wherein said first and said second surfaces are asymmetrical.

3. The improved rotary tattoo machine of claim 1 wherein said drive element provides at least one periodic motion upon the linking member during operation of said reversible motor.

4. The improved rotary tattoo machine of claim 3 wherein said periodic motion is an oscillating motion which is at least partially linear.

5. The improved rotary tattoo machine of claim 1 wherein said reversible motor is operable in a clockwise direction and a counter-clockwise direction.

6. The improved rotary tattoo machine of claim 5 wherein said oscillation in said clockwise direction is different from said oscillation in said counter-clockwise direction.

7. The improved rotary tattoo machine of claim 1 further comprising a follower arm engageable by said first rotating member during operation of said reversible motor.

8. The improved rotary tattoo machine of claim 1 further comprising an elongated fastener and a biasing member received by said housing wherein said elongated fastener adjusts said drive element.

9. An improved rotary tattoo machine with a reversible motor, said rotary tattoo machine comprising:
a housing;
an actuator spaced from said housing and in communication with an armature;
a rotational drive element received by said housing and in rotational communication with said armature;
said rotational drive element engaged and centrally offset from a centric cam assembly;
said centric cam assembly presenting a dampener;
said dampener limiting rotation of said centric cam assembly in rotational communication with the reversible motor; and
said dampener presenting a clipped oscillation of said armature during rotation of said centric cam assembly.

10. The improved rotary tattoo machine of claim 9, said centric cam assembly further comprising a centric cam bearing configured for circumferentially receipt of a centric cam, wherein said dampener is presented therebetween.
